(19) United States
(12) Patent Application Publication
(10) Pub. No.: US 2020/0306164 A1
(43) Pub. Date: Oct. 1, 2020

(54) PENIS NOURISHING AND INVIGORATING CREAM

(71) Applicants: Marie Jardines, Miami, FL (US); Ruben Alcoba, Miami, FL (US)

(72) Inventors: Marie Jardines, Miami, FL (US); Ruben Alcoba, Miami, FL (US)

(21) Appl. No.: 16/364,242

(22) Filed: Mar. 26, 2019

(51) Int. Cl. A61K 8/67; A61K 8/81; A61K 8/368; A61K 8/36; A61K 8/362; A61K 8/34; A61K 8/41; A61K 8/31; A61K 8/92; A61K 8/35; A61K 8/44; A61K 8/9789; A61K 8/9794; A61Q 19/08; A61Q 19/00

(52) U.S. Cl. CPC A61K 8/676; A61Q 19/007; A61K 8/678; A61K 8/671; A61K 8/8147; A61K 8/368; A61K 8/361; A61K 8/362; A61K 8/345; A61K 8/41; A61K 8/31; A61K 8/92; A61K 8/355; A61K 8/44; A61K 8/9789; A61K 8/9794; A61K 8/347; A61Q 19/08; A61K 8/675

(57) ABSTRACT

A penis nourishing and invigorating cream that nourishes the dermis of a penis and promotes the widening of the arteries and blood vessels of the penis. The cream comprises of a distilled water, at least one antioxidant, at least on thickening agent, at least one preservative, at least one emulsifier, a skin penetrator, a mineral oil, and a nitric oxide generating natural supplement.

### BACKGROUND

The present invention is directed to a penis nourishing and invigorating cream.

As males age, the dermis of the penis suffers from normal wear and tear caused by age and activity. The dermis can develop an appearance of being dry and rough looking. The appearance may be caused by an active sexual lifestyle. The dermis of the penis, similar to dermis of the rest of the human body, requires moisturizers to help maintain its appearance pleasant.

Erectile dysfunction (hereinafter ED) is sometimes caused by clogged arteries. To get and keep an erection, blood needs to flow cleanly through your penis. If you have ED, it can mean that one or more of your blood vessels have narrowed or are blocked and are therefore preventing blood flow from flowing cleanly through your penis. Blood vessels in your penis may have constricted due to plaque buildup. The present invention may promote the widening of the arteries and blood vessels of your penis.

Erections occur when the penis is flooded with blood due to the widening of the arteries. Arteries have to receive nutrients to allow them widen or dilate when need be.

The present invention provides the penis with a moisturizing cream that is comprised of antioxidants that will help the dermis of the penis maintain a pleasant and youthful appearance.

The moisturizing cream of the present invention further contains at least one compound that will allow the arteries of the penis to vasodilate. The vasodilation of the arteries of the penis will reduce the resistance of the blood flow passing through the arteries of the penis and will invigorate the penis.

The present invention provides nutritional supplements to the penis that will promote nitric oxide production within the penis. Nitric oxide causes blood vessels to open wider for improved blood flow.¹ ¹Available at https://medlineplus.gov/druginfo/natural/875.html, Natural Medicines Comprehensive Database Consumer Version, L-Arginine, seen on Mar. 25, 2019.

The nutritional supplements that will promote nitic oxide promotion will reach the arteries or blood vessels of the penis using natural skin penetrating compounds.

For the foregoing reasons there is a need for a penis nourishing and invigorating cream that will promote a pleasant appearance of a penis and promote the widening of the arteries and blood vessels of the penis.

### SUMMARY

The present invention is a penis nourishing and invigorating cream that nourishes the dermis of a penis and promotes the widening of the arteries and blood vessels of the penis. The cream comprises of distilled water, at least one antioxidant, at least on thickening agent, at least one preservative, at least one emulsifier, a skin penetrator, a mineral oil, and a nitric oxide generating natural supplement.

Antioxidants are man-made or natural substances that may prevent or delay some types of cell damage.² The antioxidants are used to nourish the dermis of the penis. ²Available at https://nccih.nih.gov/health/antioxidants/introduction.htm, NCCIH Pub No.: D483, seen on Mar. 26, 2019.

The nitric oxide generating supplements are used to promote the widening of the arteries and blood vessels of the penis.

The skin penetrating compound is used to penetrate the dermis and allow the antioxidants and the nitric oxide generating natural supplements to reach the interior arteries and blood vessels of the penis.

An object of the present invention is to provide a cream that may prolong and restore the pleasant appearance of a penis.

Another object of the present invention is to provide a skin penetrating cream that will promote nitric oxide generation within the penis.

Yet another object of the present invention is to provide a cream that will enhance (make more attractive) the outer appearance of a penis.

Still another object of the present invention is to provide a cream that may enhance (increase) the blood flow through and within the penis.

### DESCRIPTION

The present invention is a penis nourishing and invigorating cream. The cream comprises of a distilled H²O, at least one antioxidant, at least on thickening agent, at least one preservative, at least one emulsifier, a skin penetrator, a mineral oil, and a nitric oxide generating natural supplement.

Every oz of the cream will be comprised of the elements or compounds described below in the quantities described below.

The distilled H²O is in the range from about 0.8925 oz to about 0.9145 oz.

The at least one antioxidant can be selected from the group consisting of Vitamin C, Vitamin B³, Vitamin E, or Vitamin A. In an embodiment of the present invention, the Vitamin C present in the cream is in the range from about 0.0005 oz to about 0.0015 oz, the Vitamin B³ present in the cream is in the range from about 045 oz to about 0.0055 oz, the Vitamin E present in the cream is in the range from about 0.0025 oz to about 0.0035 oz, and the Vitamin A present in the cream is in the range from about 0.0015 oz to about 0.0025 oz.

The at least on thickening agent is Carbomer or any equivalent compound thereof. In a preferred embodiment, the Carbomer present in the cream is in the range from about 0.0035 oz to about 0.0045 oz.

The at least one preservative is selected from the group consisting of Sodium Benzoate, Sodium Sorbate, SALIGUARD PG or Caprylic/Capric Triglyceride. In a preferred embodiment, the Sodium Benzoate present in the cream is in the range from about 0.0025 oz to about 0.0035 oz, the Sodium Sorbate present in the cream is in the range from 0.0025 oz to about 0.0035 oz, SALIGUARD PG present in the cream is in the range from about 0.0025 oz to about 0.0035 oz, and the Caprylic/Capric Triglyceride present in the cream is in the range from about 0.0025 oz to about 0.0035 oz.

The at least one emulsifier is selected from the group consisting of Propylene Glycol or Triethanolamine. The Propylene Glycol present in the cream is in the range from about 0.0095 oz to about 0.0105 oz and the Triethanolamine present in the cream is in the range from about 0.0035 oz to about 0.0045 oz.

The skin penetrator is D Limonene or any compound equivalent thereof that can help any other compound penetrate the dermis. The D-limonene present in the cream is in the range from about 0045 oz to about 0.005 oz.

The mineral oil present in the cream is in the range from about 0.0075 oz to about 0.0085 oz.

The nitric oxide generating natural supplement is selected from the group consisting of CoQ10, L-Arginine, Cocoa, L-Citrulline or watermelon seed oil. The CoQ10 present in the cream is in the range from about 0.0004 oz to about 0.0005 oz, the L-Arginine present in the cream is in the range from about 0.0095 oz to 0.0105 oz; the Cocoa powder present in the cream is in the range from about 0.0140 oz to about 0.0155 oz, and watermelon seed oil present in the cream is in the range from about 0.0035 oz to about 0.0045 oz.

In a preferred embodiment of the present invention, the L-Arginine present in the cream will be in the range from 0.0095 oz to 0.0200 oz and the watermelon seed oil present in the cream is in the range from about 0.0035 oz to about 0.0095 oz. In this embodiment, the Cocoa powder would not be present in this embodiment of the cream.

An embodiment of the cream may comprise of a Black Maca powder. The Black Maca powder is in the range from about 0.0095 oz to about 0.0105 oz.

In a further embodiment, the cream may comprise of a vanilla oil and a cinnamon oil. The vanilla oil and the cinnamon oil are placed in the cream to provide a possible aromatic aphrodisiac to the cream. In a preferred embodiment, the vanilla oil present in the cream is in the range from about 0035 oz to about 0.0045 oz and the cinnamon oil present in the cream is in the range from about 0.0001 oz to about 0.0010 oz.

In still another embodiment, the cream may comprise of Pomegranate seed oil, Safflower oil, and Cannabidiol oil. In this embodiment, the Pomegranate seed oil present in the cream is in the range from about 0.0025 oz to about 0.0035 oz, the Safflower oil present in the cream is in the range from about 0.0025 oz to about 0.0035 oz, and the cannabidiol oil present in the cream is in the range from about 0.0025 oz to about 0.0035 oz.

An advantage of the present invention is that it provides a cream that prolongs and restores the pleasant appearance of a penis.

Another advantage of the present invention is that it provides a skin penetrating cream that promotes nitric oxide generation within the penis.

Yet another advantage of the present invention is that it provides a cream that enhances (make more attractive) the outer appearance of a penis.

Still another advantage of the present invention is that it provides a cream that enhances (increases) the blood flow through and within the penis.

While the inventor's above description contains many specificities, these should not be construed as limitations on the scope, but rather as an exemplification of several preferred embodiments thereof. Many other variations are possible. Accordingly, the scope should be determined not by the embodiments illustrated, but by the appended claims and their legal equivalents.

What is claimed is:

1. A non-medicated penis nourishing and invigorating cream, the cream comprises:
a distilled H²O;
at least one antioxidant;
at least on thickening agent;
at least one preservative;
at least one emulsifier;
a skin penetrator;
a mineral oil; and
a nitric oxide generating natural supplement.

2. The non-medicated penis nourishing and invigorating cream of claim 1, wherein:
the at least one antioxidant is selected from the group consisting of Vitamin C, Vitamin B³, Vitamin E, or Vitamin A;
the at least on thickening agent is Carbomer;
the at least one preservative is selected from the group consisting of Sodium Benzoate, Sodium Sorbate, SALIGUARD PG or Caprylic/Capric Triglyceride;
the at least one emulsifier is selected from the group consisting of Propylene Glycol or Triethanolamine;
the skin penetrator is D Limonene; and
the nitric oxide generating natural supplement is selected from the group consisting of CoQ10, L-Arginine, Cocoa, L-Citrulline or watermelon seed oil.

3. The non-medicated penis nourishing and invigorating cream of claim 2, wherein every oz of the cream has in weight:
the distilled H²O is in the range from about 0.8925 oz to about 0.9145 oz;
the Vitamin C is in the range from about 0.0005 oz to about 0.0015 oz, the Vitamin B³ is in the range from about 045 oz to about 0.0055 oz, the Vitamin E is in the range from about 0.0025 oz to about 0.0035 oz, and the Vitamin A is in the range from about 0.0015 oz to about 0.0025 oz;
the Carbomer is in the range from about 0.0035 oz to about 0.0045 oz;
the Sodium Benzoate is in the range from about 0.0025 oz to about 0.0035 oz, the Sodium Sorbate is in the range from 0.0025 oz to about 0.0035 oz, SALIGUARD PG is in the range from about 0.0025 oz to about 0.0035 oz, and the Caprylic/Capric Triglyceride is in the range from about 0.0025 oz to about 0.0035 oz;
the Propylene Glycol is in the range from about 0.0095 oz to about 0.0105 oz and the Triethanolamine is in the range from about 0.0035 oz to about 0.0045 oz;
the D-limonene is in the range from about 0045 oz to about 0.005 oz;
the mineral oil is in the range from about 0.0075 oz to about 0.0085 oz; and
the CoQ10 is in the range from about 0.0004 oz to about 0.0005 oz, the L-Arginine is in the range from about 0.0095 oz to 0.0105 oz; the Cocoa powder is in the range from about 0.0140 oz to about 0.0155 oz, and watermelon seed oil is in the range from about 0.0035 oz to about 0.0045 oz.

4. The non-medicated penis nourishing and invigorating cream of claim 3, the cream comprises a Black Maca powder.

5. The non-medicated penis nourishing and invigorating cream of claim 4, wherein the Black Maca powder is in the range from about 0.0095 oz to about 0.0105 oz.

6. The non-medicated penis nourishing and invigorating cream of claim 5, the cream comprises;
a vanilla oil; and
a cinnamon oil.

7. The non-medicated penis nourishing and invigorating cream of claim 6, wherein the vanilla oil is in the range from about 0035 oz to about 0.0045 oz and the cinnamon oil is in the range from about 0.0001 oz to about 0.0010 oz.

8. The non-medicated penis nourishing and invigorating cream of claim 7, the cream comprises;
Pomegranate seed oil;
Safflower oil; and
Cannabidiol oil.

9. The non-medicated penis nourishing and invigorating cream of claim 8, wherein the Pomegranate seed oil is in the range from about 0.0025 oz to about 0.0035 oz, the Safflower oil is in the range from about 0.0025 oz to about 0.0035 oz, and the cannabidiol oil is in the range from about 0.0025 oz to about 0.0035 oz.

10. The non-medicated penis nourishing and invigorating cream of claim 2, wherein every oz of the cream has in weight:
the distilled H2O is in the range from about 0.8925 oz to about 0.9145 oz;
the Vitamin C is in the range from about 0.0005 oz to about 0.0015 oz, the Vitamin B3 is in the range from about 045 oz to about 0.0055 oz, the Vitamin E is in the range from about 0.0025 oz to about 0.0035 oz, and the Vitamin A is in the range from about 0.0015 oz to about 0.0025 oz;
the Carbomer is in the range from about 0.0035 oz to about 0.0045 oz;
the Sodium Benzoate is in the range from about 0.0025 oz to about 0.0035 oz, the Sodium Sorbate is in the range from 0.0025 oz to about 0.0035 oz, SALIGUARD PG is in the range from about 0.0025 oz to about 0.0035 oz, and the Caprylic/Capric Triglyceride is in the range from about 0.0025 oz to about 0.0035 oz;
the Propylene Glycol is in the range from about 0.0095 oz to about 0.0105 oz and the Triethanolamine is in the range from about 0.0035 oz to about 0.0045 oz;
the D-limonene is in the range from about 0045 oz to about 0.005 oz;
the mineral oil is in the range from about 0.0075 oz to about 0.0085 oz; and
the CoQ10 is in the range from about 0.0004 oz to about 0.0005 oz, the L-Arginine is in the range from about 0.0095 oz to 0.0200 oz and watermelon seed oil is in the range from about 0.0035 oz to about 0.0095 oz.

11. The non-medicated penis nourishing and invigorating cream of claim 10, the cream comprises a Black Maca powder.

12. The non-medicated penis nourishing and invigorating cream of claim 11, wherein the Black Maca powder is in the range from about 0.0095 oz to about 0.0105 oz.

13. The non-medicated penis nourishing and invigorating cream of claim 12, the cream comprises;
a vanilla oil; and
a cinnamon oil.

14. The non-medicated penis nourishing and invigorating cream of claim 13, wherein the vanilla oil is in the range from about 0035 oz to about 0.0045 oz and the cinnamon oil is in the range from about 0.0001 oz to about 0.0010 oz.

15. The non-medicated penis nourishing and invigorating cream of claim 14, the cream comprises;
Pomegranate seed oil;
Safflower oil; and
Cannabidiol oil.

16. The non-medicated penis nourishing and invigorating cream of claim 15, wherein the Pomegranate seed oil is in the range from about 0.0025 oz to about 0.0035 oz, the Safflower oil is in the range from about 0.0025 oz to about 0.0035 oz, and the cannabidiol oil is in the range from about 0.0025 oz to about 0.0035 oz.
